(19) United States
(12) Patent Application Publication
(10) Pub. No.: US 2007/0225636 A1
(43) Pub. Date: Sep. 27, 2007

(54) Surgical suction device

(76) Inventor: Michael Hahn, Oklahoma City, OK (US)

Correspondence Address: TAYLOR & AUST, P.C., 142 SOUTH MAIN STREET, P. O. BOX 560, AVILLA, IN, 46710, US

(21) Appl. No.: 11/385,300

(22) Filed: Mar. 21, 2006

(51) Int. Cl. A61M 1/00

(52) U.S. Cl. 604/35

(57) ABSTRACT

A surgical suction device which includes a first tubular section with a first inlet end and a first outlet end. The first tubular section has a variable inside diameter which continuously expands from the first inlet end to the first outlet end. A curved tubular section is connected to the first outlet end, and the curved tubular section has a second inlet end and a second outlet end. The curved tubular section further includes a second inside diameter which extends from the second inlet end to the second outlet end, where the second inside diameter is approximately constant from the second inlet end to the second outlet end. The second inside diameter is also not less than the variable inside diameter at the first outlet end.

### BACKGROUND OF THE INVENTION

1. Field of the Invention

The present invention relates to surgical systems, and, more particularly, to a surgical suction device or nozzle.

2. Description of the Related Art

A probe and suction device for use by surgeons is known to remove blood, fluid and surgical debris from the surgical site and to permit the surgeon to probe within the surgical site. These devices typically are rigid one-piece instruments with a straight-line elongated probe. Further, such a suction device is not particularly ergonomic in that a patient is typically horizontal on an operating table, and for best use, the suction device needs to be tilted into the surgical incision which requires an awkward positioning of the surgeon's, or other surgical personnel's, hand/wrist.

A suction device is known which includes a curved section which allows for a more relaxed and natural positioning of a surgeon's hand/wrist. However, the curved section is created by bending a tube, and such bending action restricts the inside diameter of the bent portion by creating an oval-shaped cross-section of the inside diameter, for example. This restriction can create a blockage as surgical debris is drawn into the suction device. Such a blockage can delay the removal of the debris, and therefore delay the surgery, with adverse effects. Procedural steps in a surgical procedure can be very time critical, and a blocked suction device can create delays at critical moments within the procedure. Further, operating time in surgery is relatively expensive, and it is quite inefficient to have highly trained and skilled medical personnel clearing or replacing a blocked suction device during surgery.

What is needed in the art is surgical suction device which is ergonomic to use and which does not include any restrictions which can block the flow of surgical debris material.

### SUMMARY OF THE INVENTION

The present invention provides a surgical suction device with a first section having an expanding inside diameter and a following curved section having no restrictions.

The invention comprises, in one form thereof, a surgical suction device which includes a first tubular section with a first inlet end and a first outlet end. The first tubular section has a variable inside diameter which continuously expands from the first inlet end to the first outlet end. A curved tubular section is connected to the first outlet end, and the curved tubular section has a second inlet end and a second outlet end. The curved tubular section further includes a second inside diameter which extends from the second inlet end to the second outlet end, where the second inside diameter is approximately constant from the second inlet end to the second outlet end. The second inside diameter is also not less than the variable inside diameter at the first outlet end.

The invention comprises, in another form thereof, a surgical system which includes a vacuum system and a surgical suction device connected to the vacuum system. The surgical suction device includes a first tubular section with a first inlet end and a first outlet end. The first tubular section has a variable inside diameter which continuously expands from the first inlet end to the first outlet end. A curved tubular section is connected to the first outlet end, and the curved tubular section has a second inlet end and a second outlet end. The curved tubular section further includes a second inside diameter which extends from the second inlet end to the second outlet end, where the second inside diameter is approximately constant from the second inlet end to the second outlet end. The second inside diameter is also not less than the variable inside diameter at the first outlet end.

The invention comprises, in yet another form thereof, a method of manufacturing a surgical suction device, including the steps of: forming a first tubular section having a first inlet end and a first outlet end, and a variable inside diameter which continuously expands from the first inlet end to the first outlet end; shaping a second tubular section into a curved tubular section including a second inlet end and a second outlet end, the curved tubular section having a second inside diameter extending from the second inlet end to the second outlet end, the curved tubular section being shaped such that the second inside diameter is approximately constant from the second inlet end to the second outlet end, the second inside diameter being not less than the variable inside diameter at the first outlet end; and connecting the first outlet end to the curved tubular section.

An advantage of the present invention is that it provides an ergonomic surgical suction device.

Another advantage of the present invention is that it provides a surgical suction device without internal restrictions.

Yet another advantage of the present invention is that it minimizes internal blockages of surgical debris.

### BRIEF DESCRIPTION OF THE DRAWINGS

The above-mentioned and other features and advantages of this invention, and the manner of attaining them, will become more apparent and the invention will be better understood by reference to the following description of an embodiment of the invention taken in conjunction with the accompanying drawings, wherein:

FIG. 1 is a perspective view of an embodiment of a surgical system including a surgical suction device according to the present invention;

FIG. 2 is a top view of the surgical suction device of FIG. 1;

FIG. 3 is a cross-sectional view of the surgical suction device taken along section line 3-3 in FIG. 2;

FIG. 4 is an expanded cross-sectional view of detail A of FIG. 3;

FIG. 5 is a side view of the surgical suction device of FIG. 1, shown in surgical use just prior to suctioning surgical debris in a surgical incision; and

FIG. 6 is a partially cross-sectional side view of the surgical suction device of FIG. 1, shown in surgical during the suctioning of surgical debris in a surgical incision.

Corresponding reference characters indicate corresponding parts throughout the several views. The exemplification set out herein illustrates one preferred embodiment of the invention, in one form, and such exemplification are not to be construed as limiting the scope of the invention in any manner.

### DETAILED DESCRIPTION OF THE INVENTION

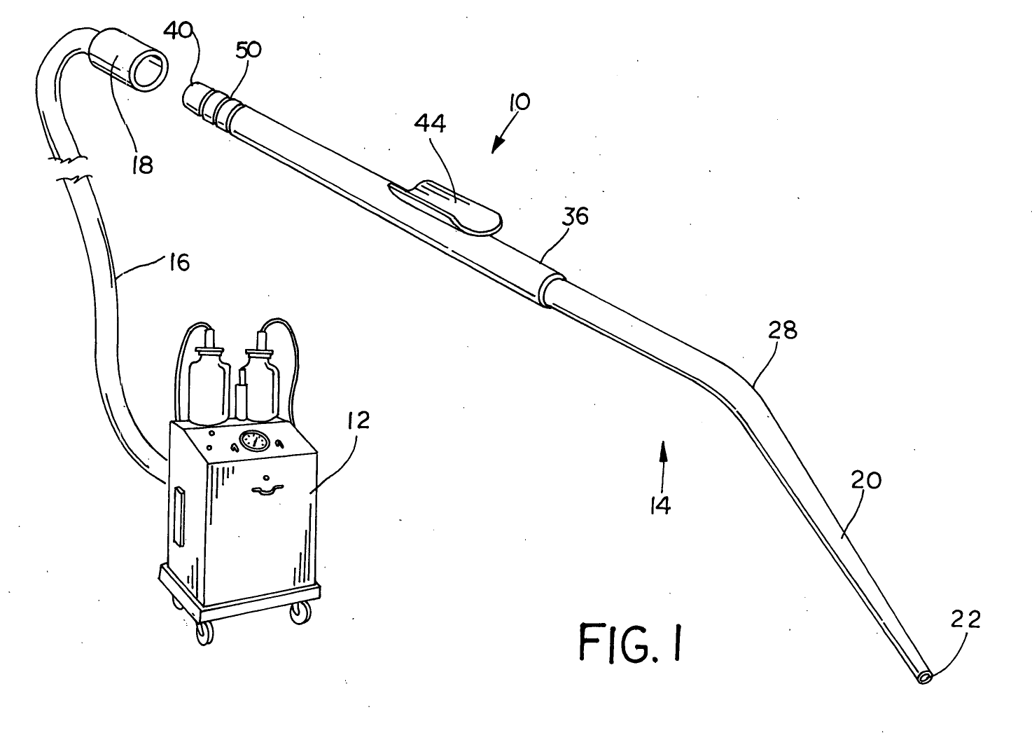

Referring now to the drawings, and more particularly to FIG. 1, there is shown a surgical system 10 which generally includes a vacuum system 12, and a surgical suction device 14 connected to vacuum system 10 with hose 16. Hose 16 can include a fitting 18 which connects to surgical suction device 14. Vacuum system 12 can be an aspirator, for example.

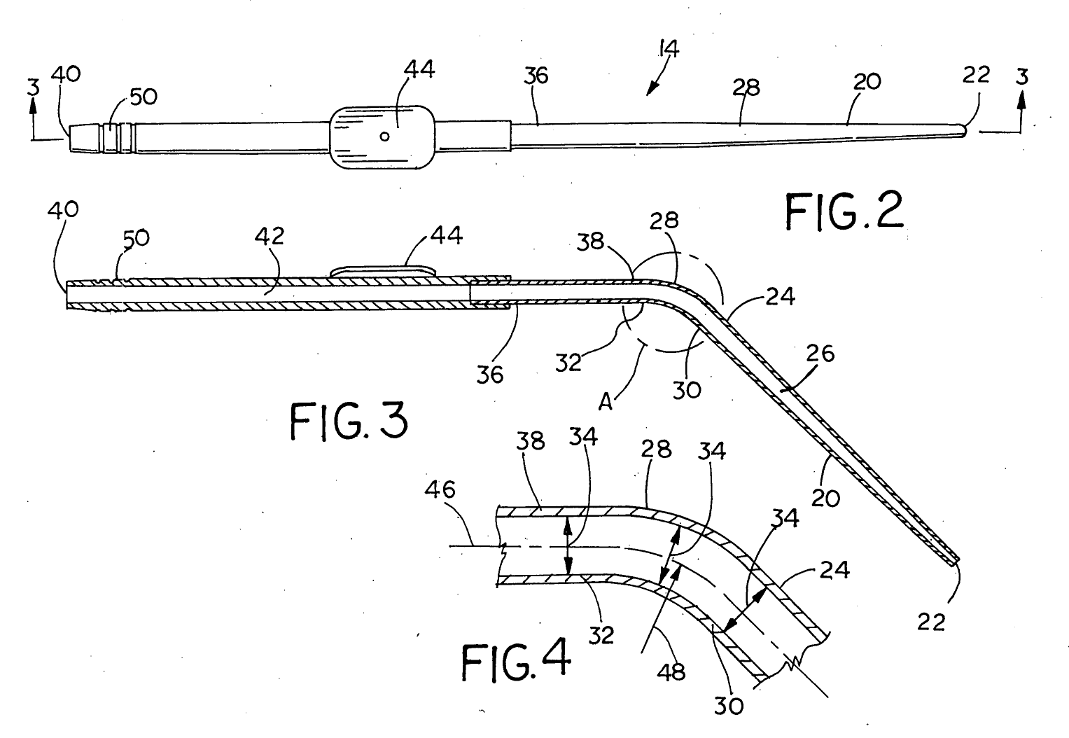

Surgical suction device 14, shown more particularly in FIGS. 2-4, can include a first tubular section 20 which has a first inlet end 22 and a first outlet end 24. First tubular section 20 includes a variable inside diameter 26 which continuously expands from first inlet end 22 to first outlet end 24. A curved tubular section 28 is connected to first outlet end 24, and curved tubular section 28 includes a second inlet end 30 and a second outlet end 32. Curved tubular section 28 includes a second inside diameter 34 extending from second inlet end 30 to second outlet end 32. Second inside diameter 34 is approximately constant, or constant, or expanding from second inlet end 30 to second outlet end 32. Second inside diameter 34 is not less than variable inside diameter 26 at first outlet end 24.

Variable inside diameter 26 can expand linearly from first inlet end 22 to first outlet end 24. A third tubular section 36 is connected to second outlet end 30. Third tubular section 36 has a third inlet end 38, a third outlet end 40 and a third inside diameter 42 extending from third inlet end 38 and third outlet end 40. Third inside diameter 42 is not less than variable inside diameter 26 at first outlet end 24.

A finger rest 44 is connected to third tubular section 36. Finger rest 44 can be in a form of a curved plate as shown. Finger rest 44 can be tangentially connected to an outside of third tubular section 36, also as shown. Curved tubular section 28 can have a centerline 46 extending from second inlet end 30 to second outlet end 32, and centerline 46 can include an approximately constant radius 48 of curvature.

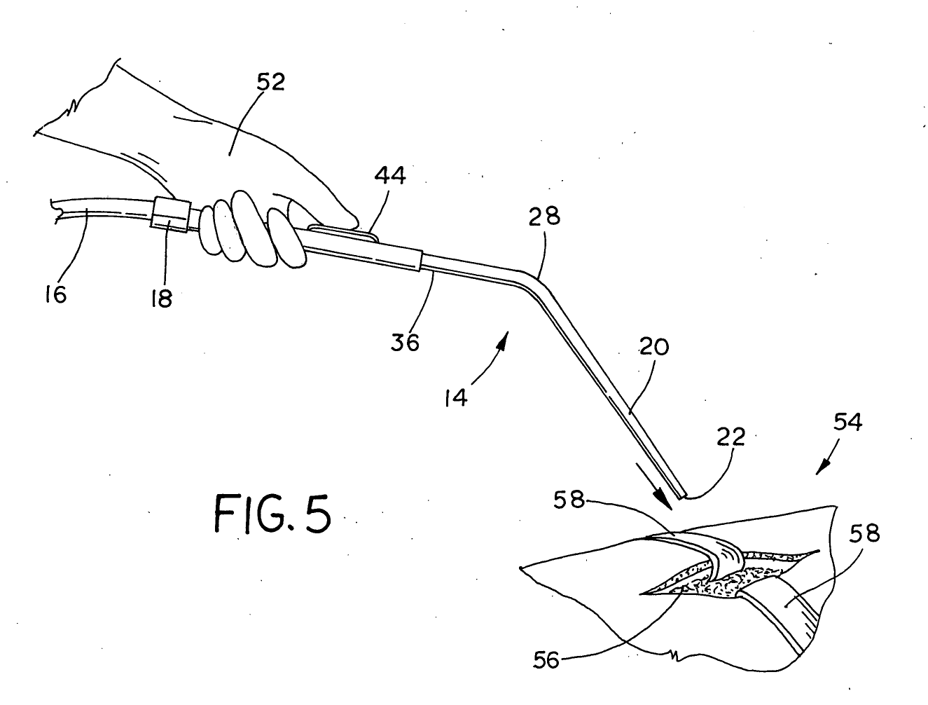

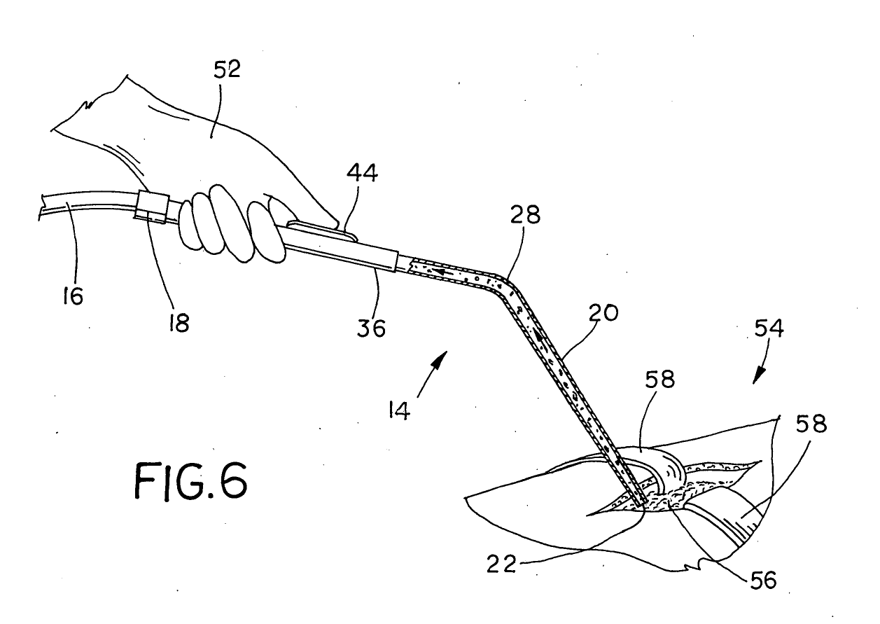

Surgical suction device 14 can include fitting compatible structure at 50 for connection to hose fitting 18, and hose 16 can be connected to aspirator 12. FIGS. 5 and 6 illustrate how a surgeon 52, or surgical personnel, can use surgical suction device 14 to aspirate a surgical site 54, in particular an incision 56 held open by retractors 58.

In use, the present invention discloses a method of manufacturing a surgical suction device, comprising the steps of: forming first tubular section 20 including first inlet end 22 and first outlet end 24, first tubular section 20 including variable inside diameter 26 which continuously expands from first inlet end 22 to first outlet end 24; shaping a second tubular section 28 into curved tubular section 28 including a second inlet end 30 and a second outlet end 32, and a second inside diameter 34 extending from second inlet end 30 to second outlet end 32, curved tubular section 28 being shaped such that second inside diameter 34 is approximately constant from second inlet end 30 to second outlet end 32, second inside diameter 34 being not less than variable inside diameter 26 at first outlet end 24; and connecting first outlet end 24 to curved tubular section 28. The method can further include the step of connecting third tubular section 36 to second outlet end 32, third tubular section 36 having third inlet end 38, third outlet end 40 and third inside diameter 42 extending from third inlet end 38 and third outlet end 40, third inside diameter 42 not less than variable inside diameter 26 at first outlet end 24. The method can further include the steps of forming finger rest 44 in a form of a curved plate; and connecting finger rest 44 to third tubular section 36. The shaping step can include a bending operation.

While this invention has been described as having a preferred design, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A surgical suction device, comprising:
a first tubular section including a first inlet end and a first outlet end, said first tubular section including a variable inside diameter which continuously expands from said first inlet end to said first outlet end; and
a curved tubular section connected to said first outlet end, said curved tubular section including a second inlet end and a second outlet end, said curved tubular section including a second inside diameter extending from said second inlet end to said second outlet end, said second inside diameter being approximately constant from said second inlet end to said second outlet end, said second inside diameter being not less than said variable inside diameter at said first outlet end.

2. The surgical suction device of claim 1, wherein said variable inside diameter expands linearly from said first inlet end to said first outlet end.

3. The surgical suction device of claim 1, further including a third tubular section connected to said second outlet end, said third tubular section having a third inlet end, a third outlet end and a third inside diameter extending from said third inlet end and said third outlet end, said third inside diameter not less than said variable inside diameter at said first outlet end.

4. The surgical suction device of claim 3, further including a finger rest connected to said third tubular section.

5. The surgical suction device of claim 4, wherein said finger rest is in a form of a curved plate.

6. The surgical suction device of claim 5, wherein said finger rest is tangentially connected to an outside of said third tubular section.

7. The surgical suction device of claim 1, wherein said curved tubular section has a centerline extending from said second inlet end to said second outlet end, said centerline includes an approximately constant radius of curvature.

8. A surgical system, comprising:
a vacuum system;
a surgical suction device connected to said vacuum system, said surgical suction device including:
a first tubular section including a first inlet end and a first outlet end, said first tubular section including a variable inside diameter which continuously expands from said first inlet end to said first outlet end; and
a curved tubular section connected to said first outlet end, said curved tubular section including a second inlet end and a second outlet end, said curved tubular section 10 including a second inside diameter extending from said second inlet end to said second outlet end, said second inside diameter being approximately constant from said second inlet end to said second outlet end, said second inside diameter being not less than said variable inside diameter at said first outlet end.

9. The surgical system of claim 8, wherein said variable inside diameter expands linearly from said first inlet end to said first outlet end.

10. The surgical system of claim 8, further including a third tubular section connected to said second outlet end, said third tubular section having a third inlet end, a third outlet end and a third inside diameter extending from said third inlet end and said third outlet end, said third inside diameter not less than said variable inside diameter at said first outlet end.

11. The surgical system of claim 10, further including a finger rest connected to said third tubular section.

12. The surgical system of claim 11, wherein said finger rest is in a form of a curved plate.

13. The surgical system of claim 12, wherein said finger rest is tangentially connected to an outside of said third tubular section.

14. The surgical system of claim 8, wherein said curved tubular section has a centerline extending from said second inlet end to said second outlet end, said centerline includes an approximately constant radius of curvature.

15. The surgical system of claim 8, wherein said vacuum system is an aspirator.

16. A method of manufacturing a surgical suction device, comprising the steps of:
forming a first tubular section including a first inlet end and a first outlet end, said first tubular section including a variable inside diameter which continuously expands from said first inlet end to said first outlet end;
shaping a second tubular section into a curved tubular section including a second inlet end and a second outlet end, and a second inside diameter extending from said second inlet end to said second outlet end, said curved tubular section being shaped such that said second inside diameter is approximately constant from said second inlet end to said second outlet end, said second inside diameter being not less than said variable inside diameter at said first outlet end; and
connecting said first outlet end to said curved tubular section.

17. The method of claim 16, further including the step of connecting a third tubular section to said second outlet end, said third tubular section having a third inlet end, a third outlet end and a third inside diameter extending from said third inlet end and said third outlet end, said third inside diameter not less than said variable inside diameter at said first outlet end.

18. The method of claim 17, further including the step of forming a finger rest in a form of a curved plate.

19. The method of claim 18, further including the step of connecting said finger rest to said third tubular section.

20. The method of claim 16, wherein said shaping step includes a bending operation.
